(19) United States
(12) Patent Application Publication
(10) Pub. No.: US 2021/0267786 A1
(43) Pub. Date: Sep. 2, 2021

(54) PATIENT HAND IMMOBILIZER

(71) Applicant: Garry Kitay, Jacksonville, FL (US)

(72) Inventor: Garry Kitay, Jacksonville, FL (US)

(21) Appl. No.: 17/188,699

(22) Filed: Mar. 1, 2021

Related U.S. Application Data
(60) Provisional application No. 62/983,902, filed on Mar. 2, 2020.

(51) Int. Cl. A61F 5/37

(52) U.S. Cl. CPC A61F 5/3761

(57) ABSTRACT

A hand immobilizer for use during operations on the hand of the patient to extend the fingers and permit easier access to the palm and volar fingers in order to perform procedures without the fingers flexing and obstructing the field of surgery is disclosed.

### BACKGROUND OF THE INVENTION

The present invention relates to hand immobilizers, such as disclosed in U.S. Pat. No. 5,560,375 to Kabanek. The hand immobilizer is used during operations on the hand of the patient to extend the fingers and permit easier access to the palm and volar fingers in order to perform procedures without the fingers flexing and obstructing the field of surgery. The hand immobilizer is in the general shape of the hand and fingers. The prior art Kabanek patent discloses flexible tab elements at the top of the finger portions for bending over the very top of the fingers, best shown in FIG. 3 of the Kabanek patent. However, the Kabanek hand immobilizer permits the fingers of the patient to slip out from under the flexible foldable tab elements, limiting its usefulness.

### SUMMARY OF THE INVENTION

The present invention is an improvement of the Kabanek device and includes flexible tab elements extending generally perpendicularly from the finger tips so that the side tab elements can wrap around the digits, thereby further preventing the fingers of the patient from slipping out from under the finger holder. It is possible, but unnecessary, to have flexible tab elements from the top, as in Kabanek to also be present. In an alternative embodiment, a single flexible tab extends from the side.

### OBJECTS OF THE INVENTION

It is an object of the present invention to provide an improved hand immobilizer;

It is another object of the present invention to provide a hand immobilizer that is easy to use;

It is another object to provide a hand immobilizer that is easy to manufacture;

These and other objects of the present invention will be apparent from a review of the accompanying drawings and specifications.

### BRIEF DESCRIPTION OF THE DRAWINGS

FIG. 1 is a back view of the hand immobilizer of the present invention;

FIG. 2 is a front view of the present invention;

FIG. 3 is a front view of the present invention with a flexible tab bent over the fingers of the patient;

FIG. 4 is a side view of the present invention with a tab element bent over one of the fingers of the patient;

FIG. 5 is an alternative embodiment of the present invention.

FIG. 6 is a side view of an alternative embodiment of the present invention which is usable for both right and left hands.

### DETAILED DESCRIPTION OF THE DRAWINGS

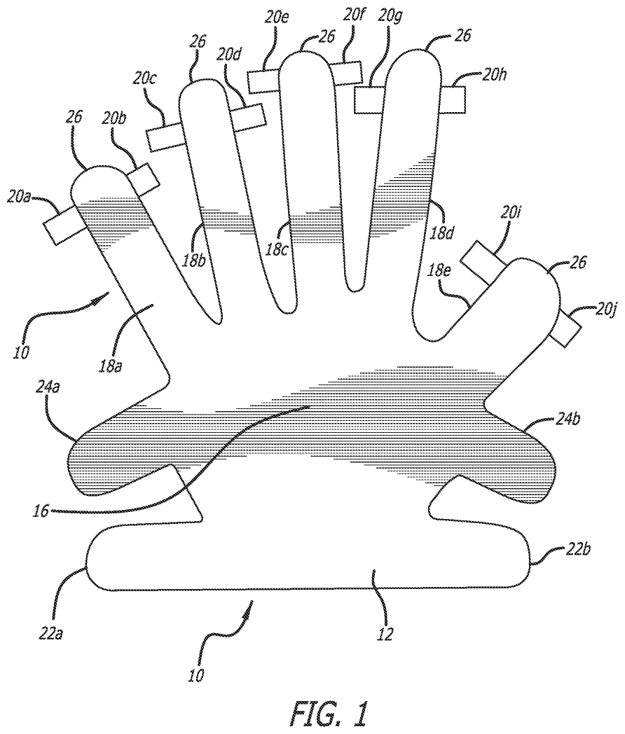

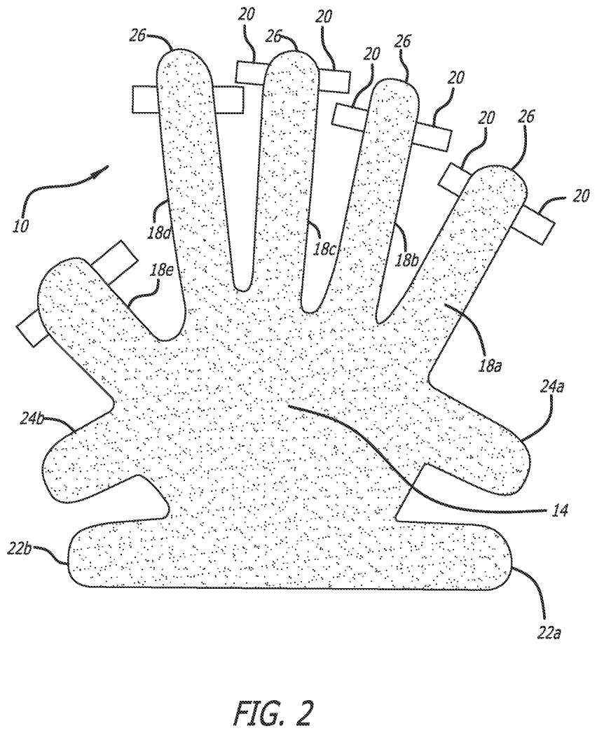

Referring to FIGS. 1 and 2, the entire article is indicated at 10 and includes two layers, a layer of aluminum 12 and a layer of sponge material 14 forming a cushion. The sheets 12 and 14 are glued together by suitable glue.

The final article or product is shaped as shown in FIGS. 1 and 2, and includes a main segment 16 corresponding to the main part of the hand, or metacarpus and finger segments 18(a)-18(e). It also includes a pair of digital segments 20 extending generally perpendicular to the central axis of each of the finger segments 18 proximate the tip 26 of the finger segment 18 corresponding to the fingers of the hand, and side tab segments 22 to be identified in detail herein below. The individual finger segment tabs are referred to as 20a-20j.

A pair of side tab segments pair 22a and 22b extending laterally outward at the bottom and a pair of side tab segments 24a and 24b, extending an angle proximate the palm of a hand. The segments 20-24 are all flexible enough to wrap around the hand and retain their position when wrapped around the hand.

In the preferred embodiment, the projecting tabs are 1 inch long and ½ inch wide. As shown in FIG. 1, the pairs of tabs 20b and 20c; 20d and 20e; 20f and 20g are offset from one another so that the two pairs of tab segments adjacent the finger segments 18a, 18b, 18c, and 18d do not overlap one another.

As shown in FIG. 5, an alternate embodiment of the present invention a single tab segment 28 is shown proximate the tip 26 end instead of two tab segments associated with each finger segment 18 as shown in FIGS. 1 and 2. In the alternative embodiment, the single segment tab 28 would be bent over the finger of the patient during use to prevent movement of the finger. The single tab 28 could be longer than the other segment tab 20.

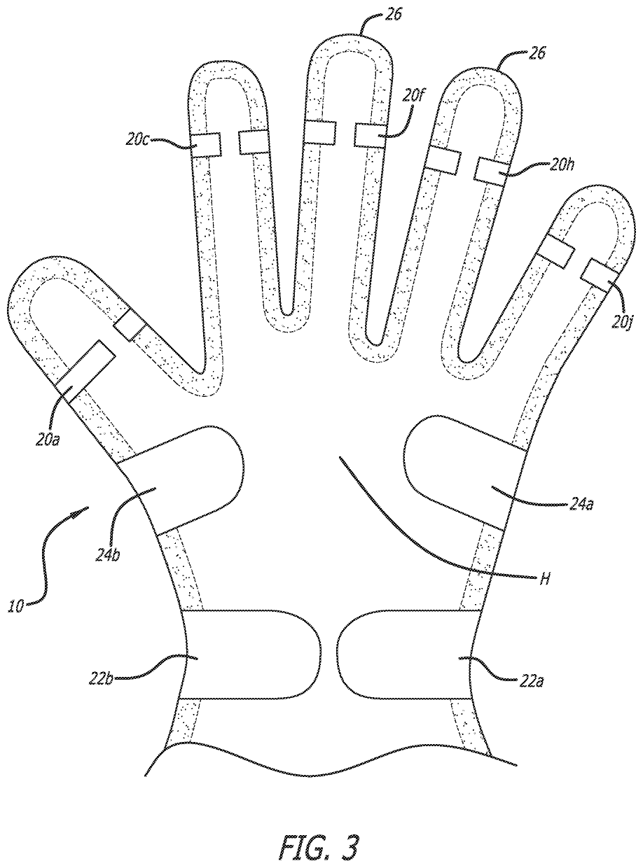

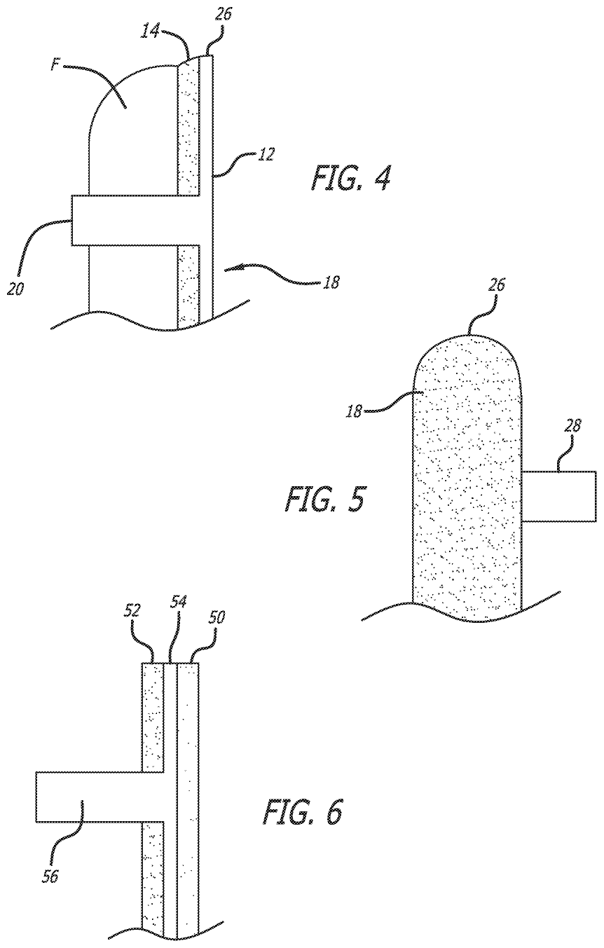

Referring to FIGS. 3 and 4, the article is applied to the patient's hand (H). In FIG. 2, the front side, or cushioned side, is shown, and the patient's hand is placed in position shown in FIG. 3, with palm side up. Then the side tab segments 22a and 22b are bent up and over the wrist portion of the hand. The innermost segments 22a and 22b are bent over the position of the hand corresponding to the wrist of the patient.

The tab segments 20-24 are folded inwardly, and thus they snugly hold the article 10 from moving or sliding off the hand.

FIG. 4 shows one of the tab elements 20 folded over one of the fingers F of the hand.

The article is of sufficient size that when it is applied to the hand, the ends of the digits of the hand reach near to tips 26 of the digital segments 18a-18e, and the tabs 20 are proximate the ends 26 of each of those digital segments.

The aluminum layer is of relatively great strength so that forces such as those tending to double up the hand, will not deflect those tabs, so as to enable the fingertips to be released. While the dimension of the aluminum layer may be any within a substantial, range, it is found that a thickness of 0.034″ is satisfactory. The sponge layer 14 may be for example of 0.7/32″ in thickness. As will be obvious, these dimensions are not limiting, but indicative. The article can be removed from the patient's hand quite readily, by releasing the tabs by gripping them and bending them back toward straight position.

Referring to FIG. 6, a side view of the invention is shown wherein a sponge layer 50 and 52 are applied to each side of the aluminum layer 54 so that the hand can be applied to either side of the invention depending if it is to be used for the right hand or the left hand. The tabs 56 can be bent in either direction.

While the present invention has been described in detail with regards to the preferred embodiments, it is appreciated that other variations of the present invention may be devised which do not depart from the inventive concept of the present invention.

1. An article constituted by a patient's hand immobilizer, the hand constituting the terminal portion of the limb, comprising,
a rigid panel to be fitted to the hand and having an inner end and an outer end,
the panel being integral and of one-piece of aluminum, and being planar in shape, and of uniform thickness throughout its outline dimensions,
the panel including
a) a main segment shaped to the metacarpus,
b) digital segments shaped to the fingers respectively and positioned correspondingly thereto, and
c) at least one flexible tab element extending generally perpendicular to the central axis of the digital segments.
the tab elements bendable over the sides of the fingers of the patient for securing the article on the hand.

2. The article of claim 1 including a pair of flexible tab elements extending from each digital segment shaped to the fingers.

3. The article of claim 2 in which the flexible tab elements on one finger are offset from one another.
